(19) United States
(12) Patent Application Publication
(10) Pub. No.: US 2015/0297678 A1
(43) Pub. Date: Oct. 22, 2015

(54) COMPOSITIONS AND METHODS OF ENHANCING WEIGHT GAIN

(71) Applicant: Wake Forest University Health Sciences, Winston-Salem, NC (US)

(72) Inventor: Emmanuel C. Opara, Durham, NC (US)

(21) Appl. No.: 14/380,624

(22) PCT Filed: Feb. 27, 2013
(86) PCT No.: PCT/US13/28001

§ 371 Date: Aug. 22, 2014

Related U.S. Application Data
(60) Provisional application No. 61/606,016, filed on Mar. 2, 2012.

(51) Int. Cl. A61K 38/18; A61K 35/39; A61K 9/50; A61K 38/28

(52) U.S. Cl. CPC A61K 38/1825; A61K 38/28; A61K 35/39; A61K 9/50; A61K 9/5052

(57) ABSTRACT

The present invention generally relates to compositions and methods of enhancing weight gain and/or myogenesis in a subject (e.g., a subject afflicted with cachexia) by the administration of fibroblast growth factor.

### STATEMENT OF GOVERNMENT SUPPORT

This invention was made with government support under Grant No. 1RO1DK080897-01A2. The United States government has certain rights to this invention.

### FIELD OF THE INVENTION

The present invention generally relates to compositions and methods of enhancing weight gain and/or myogenesis in a subject.

### BACKGROUND OF THE INVENTION

Cachexia, also known as “wasting syndrome,” is a loss of body mass that cannot be effectively treated nutritionally. It is seen in patients with cancer, AIDS, chronic obstructive lung disease, and other conditions. It is a positive risk factor for death, and few treatments are available. Hence, new compositions and methods are needed to enhance weight gain in a subject. The present invention addresses previous shortcomings in the art by providing compositions and methods of enhancing weight gain and/or myogenesis in a subject.

### SUMMARY OF THE INVENTION

One aspect of the present invention comprises a method of enhancing weight gain in a subject in need thereof, comprising: administering said subject a fibroblast growth factor (FGF) in an amount effective to enhance weight gain in said subject.

A second aspect of the present invention comprises a method of enhancing myogenesis in a subject, comprising: administering said subject a fibroblast growth factor (FGF) in an amount effective to enhance myogenesis in said subject.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1 shows weight gain in rats treated with insulin via implanted islet cells, with and without concurrent administration of FGF-1.

FIG. 2 shows weight gain in rats treated with FGF-1, with and without the concurrent administration of insulin via implanted islet cells.

### DETAILED DESCRIPTION OF ILLUSTRATIVE EMBODIMENTS

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976); see also MPEP §2111.03. Thus, the term “consisting essentially of” as used herein should not be interpreted as equivalent to “comprising.”

The term “about,” as used herein when referring to a measurable value such as an amount or concentration (e.g., the amount of a fibroblast growth factor), is meant to encompass variations of 10%, 5%, 1%, 0.5%, or even 0.1% of the specified amount.

The present invention finds use in both veterinary and medical applications. Suitable subjects of the present invention include, but are not limited to mammals. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), and mammals in utero. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In some embodiments of the present invention, the subject is a female, particularly a menopausal female.

In particular embodiments of the present invention, the subject is “in need of” the methods of the present invention, e.g., the subject has been diagnosed with a disease or disorder, the subject is at risk for a disease or disorder, or it is believed that the subject has a disease or disorder. In some embodiments of the present invention, the subject has been diagnosed with diabetes or is at risk for diabetes. Where the subject or patient has not been diagnosed with diabetes, they may be in need of treatment for cachexia, such as cachexia in a patient with cancer, acquired immune deficiency syndrome (AIDS), chronic obstructive lung disease, multiple sclerosis, congestive heart failure, tuberculosis, familial amyloid polyneuropathy, kidney failure, mercury poisioning, autoimmune disorders, and other hormonal deficiencies. The patient or subject may also be afflicted with malabsorption syndrome (such as in Crohn's disease or celiac disease). In other embodiments of the present invention, the subject has been diagnosed with a disorder that benefits from hormone replacement therapy or is at risk for a disorder that benefits from hormone replacement therapy.

“Treat,” “treating” or “treatment of” (and grammatical variations thereof) as used herein refers to any type of treatment that imparts a benefit to a subject and can mean that the severity of the subject's condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

“Pharmaceutically acceptable” as used herein means that the compound or composition is suitable for administration to a subject to achieve the treatments described herein, without unduly deleterious side effects in light of the severity of the disease and necessity of the treatment.

“Biologically active compound” as used herein may be any suitable compound, including but not limited to TGF-beta, epithelial growth factor (EGF), insulin-like growth factor-1 (IGF-1), transforming growth factors alpha and beta (TGF-1 alpha and beta), fibroblast growth factor (e.g., FGF-1, FGF-2, etc.), nerve growth factor (NGF), platelet-derived growth factor (PDGF), vascular endothelial growth factor/vascular permeability factor (VEGF/VPF), anti-virals, anti-bacterials, anti-inflammatory, immuno-suppressants, analgesics, vascularizing agents or pro-angiogenic agents, and cell adhesion molecules, and combinations thereof. See, e.g., US Patent Application No, 20110052715 (Mar. 3, 2011).

### 1. Fibroblast Growth Factor.

One aspect of the present invention provides a method of enhancing weight gain in a subject, optionally afflicted with diabetes, the method comprising administering a fibroblast growth factor (FGF) to a subject in an amount effective to enhance weight gain in the subject. In some embodiments of the present invention, two or more different fibroblast growth factors are administered to a subject. In particular embodiments of the present invention, a fibroblast growth factor is fibroblast growth factor-1 (FGF-1) and/or fibroblast growth factor-2 (FGF-2).

Fibroblast growth factor can be obtained from any suitable source, such as a mammal, particularly a human. A fibroblast growth factor can comprise a fragment of at least about 10, 15, 20, 25, 35, 50, 75, 100, 150 or more consecutive amino acids of a fibroblast growth factor. In particular embodiments of the present invention, a fibroblast growth factor is biologically active. A “biologically active” fibroblast growth factor is one that substantially retains at least one biological activity normally associated with the wild-type (i.e., native) fibroblast growth factor. In particular embodiments of the present invention, a biologically active fibroblast growth factor substantially retains all of the biological activities possessed by the wild-type (e.g., native) fibroblast growth factor. By “substantially retains” biological activity, it is meant that the fibroblast growth factor retains at least about 50%, 60%, 75%, 85%, 90%, 95%, 97%, 98%, 99%, or more, of the biological activity of the native fibroblast growth factor (and can even have a higher level of activity than the native fibroblast growth factor).

In certain embodiments of the present invention, a fibroblast growth factor can bind to heparin, promote migration, proliferation and/or differentiation of one or more cell types, bind to a FGF cell surface receptor, and/or any combination thereof. Exemplary fibroblast growth factors include, but are not limited to, those described in U.S. Pat. Nos. 4,956,455, 5,387,673, 6,451,303, 6,982,170 and U.S. Patent Application Publication No. 2004/0214759, which are incorporated by referenced in their entirety herein. The FGF may be extended activity recombinant human FGF-1 (having N-terminal His-tag) commercially available from KeraFAST Inc., 27 Drydock Ave., 2^{nd} Floor, Boston, Mass. 02210 USA.

Weight gain can be enhanced or increased in a subject by about 1%, 5%, 10%, 15%, 20%, 25,%, 30%, 40%, 50%, or more, or any range therein. In particular embodiments of the present invention, the methods of the present invention enhance weight gain in a subject by about 5% to about 25% compared to the subject's weight prior to administration of FGF according to the methods of the present invention. In certain embodiments of the present invention, the subject is afflicted with diabetes. In particular embodiments of the present invention, FGF is administered to a subject in an amount effective to cause the subject to gain more weight than compared to the same subject if administered insulin alone.

In some embodiments of the present invention, a method of enhancing myogenesis is provided, the method comprising administering a fibroblast growth factor (FGF) to a subject in an amount effective to increase myogenesis in the subject. “Myogenesis” as used herein refers to the formation of muscle tissue. Accordingly, the methods of the present invention can result in an increase in the rate of muscle tissue formation and/or in the amount of muscle tissue formed. The methods of the present invention can provide for an increase in myogenesis in a subject by about 1%, 5%, 10%, 15%, 20%, 25,%, 30%, 40%, 50%, or more, or any range therein. In particular embodiments of the present invention, the methods of the present invention enhance myogenesis in a subject by about 5% to about 25% compared to the subject's rate of muscle tissue formation and/or amount of muscle tissue prior to administration of FGF according to the methods of the present invention.

The methods of the present invention can further comprise administering one or more fibroblast growth factors and one or more biologically active compounds and/or therapeutic agents. When one or more additional fibroblast growth factors and/or additional components (e.g. a biologically active compound and/or therapeutic agent) are administered to a subject, the fibroblast growth factor(s) and/or additional components can be administered together in the same composition or separately by the same or a different method. Thus, the fibroblast growth factor(s) and/or additional components may be administered simultaneously (i.e., concurrently), sequentially, and/or administered as two or more events occurring within a short time period before or after each other (e.g., about ±1 day, ±12 hours, ±6 hours, ±4 hours, ±2 hours, ±1 hour, ±30 minutes, etc.). Simultaneous administration may be carried out by mixing the compounds prior to administration, or by administering the compounds at the same point in time but at different anatomic sites or using different routes of administration. In other embodiments of the present invention, simultaneous administration may be carried out by a substantially continuous release of a fibroblast growth factor and/or an additional component and another administration event occurring one or more times during the substantially continuous release. In certain embodiments of the present invention, one or more additional fibroblast growth factors and/or additional components are administered simultaneously.

In some embodiments of the present invention, the method comprises administering one or more fibroblast growth factors and/or one or more additional growth factors, such as, but not limited to, EGF, IGF-1, TGF-1, NGF, PDGF, and/or VEGF/VPF, to enhance weight gain and/or myogenesis in a subject. In other embodiments of the present invention, the method comprises administering a fibroblast growth factor and insulin.

In certain embodiments of the present invention, the method comprises administering one or more fibroblast growth factors and one or more cell types. Cells used to carry out the present invention are, in general, live mammalian cells collected from a suitable donor. Donors are, in general, mammalian (e.g., human, dog, cat, rabbit, rat, mouse, monkey, chimpanzee, horse, pig, goat, sheep). The donor may be of the same species as the subject being treated, or of a different species. In some embodiments of the present invention, the donor may be the same subject undergoing treatment, where suitable cells were harvested from the subject and stored for subsequent use. Exemplary cells include, but are not limited to pancreatic islet cells, ovarian cells (e.g., ovarian granulosa cells and/or ovarian theca cells), stem cells (e.g., mesenchymal stem cells isolated from bone marrow, muscle tissues, dermis, or combinations thereof), and any combination thereof.

In particular embodiments of the present invention, the method comprises administering one or more fibroblast growth factors (FGF) and one or more cell types to a subject in an amount effective to enhance weight gain and/or myogenesis in the subject. In particular embodiments of the present invention, the method comprises administering fibroblast growth factor-1 (FGF-1), pancreatic islet cells, and optionally one or more fibroblast growth factors and/or biologically active compounds to a subject in an amount effective to enhance weight gain and/or myogenesis in the subject. The methods of the present invention can optionally comprise administering an anticoagulant, such as, but not limited to heparin, hirudin, lepirudin, bivairudin, argatroban, dabigatran, ximelagatran, batroxobin, and/or hementin. In other embodiments of the present invention, the method comprises administering FGF-1, pancreatic islet cells, and heparin.

Fibroblast growth factor can be formulated and/or administered to a subject by any suitable means. For example, fibroblast growth factor can be mixed with a pharmaceutically acceptable carrier and/or excipient, such as sterile physiological saline solution. Further, any suitable technique, including but not limited to surgical implantation or injection (either of which may be carried out subcutaneously, intraperitoneally, intramuscularly, or into any other suitable compartment) can be used to administer FGF.

Dosage of cells optionally administered can be determined in accordance with known techniques or variations thereof that will be apparent to those skilled in the art. For comparison, in the treatment of diabetes, the International Islet Transplant Registry has recommended transplants of at least 6,000 cells per kilogram of recipient body weight, to achieve euglycemia. In the present invention, the number of cells administered will depend upon the age and condition of the subject, the particular disorder being treated, etc. In some embodiments of the present invention, from 1,000, 2,000, 3,000, or 6,000 cells per kilogram of recipient body weight, up to 20,000, 40,000 or 60,000 cells per kilogram recipient body weight, are administered.

In particular embodiments of the present invention, the methods of the present invention comprise administering a microparticle comprising one or more fibroblast growth factors, such as, but not limited to FGF-1 and/or FGF-2. “Microparticle” as used herein refers to a microcapsule and/or microbead. Any suitable microparticle, microcapsule and/or microbead may be used. See, e.g., U.S. Pat. Nos. 7,658,998; 7,534,448; 7,498,038; 6,677,313; 6,025,337; 5,869,103; etc. In some embodiments of the present invention, the microparticle is a microcapsule of the present invention, as described herein.

In certain embodiments of the present invention, fibroblast growth factor, such as, but not limited to FGF-1 and/or FGF-2, is administered to a subject in an amount from about 0.5 FGF-1/100 microcapsules to about 5 μg FGF-1/100 microcapsules, or any range therein, such as but not limited to, from about 1 μg FGF-1/100 microcapsules to about 2 μg FGF-1/100 microcapsules.

### 2. Microcapsule Production.

Microcapsules useful in the present invention optionally, but in some embodiments preferably, have at least one semipermeable membrane surrounding a cell-containing interior (preferably a hydrogel interior). The semipermeable membrane permits the diffusion of nutrients, biologically active molecules and other selected products through the surface membrane and into the microcapsule core. The surface membrane contains pores of a size that determines the molecular weight cut-off of the membrane. In some embodiments of the present invention, a microcapsule comprises encapsulates live cells. Encapsulation of live cells can be carried out in accordance with known techniques or variations thereof that will be apparent to those skilled in the art. See, e.g., U.S. Pat. Nos. 6,783,964, 6,365,385, and 6,303,355 to Opara, the disclosures of which are incorporated by reference herein in their entirety. The membrane pore size can be chosen to optionally allow for the passage of active agents secreted by a cell (e.g., insulin from pancreatic cells; estrogen, and in some embodiments progesterone, from ovarian cells; etc.) from the within the capsule to the external environment, but to exclude the entry of host immune response factors (where the encapsulated cells are not autologous). Such a semipermeable membrane is typically formed from a polycation such as a polyamine (e.g., polylysine and/or polyornithine), as discussed further below.

In one non-limiting example embodiment of an encapsulation technique, U.S. Pat. No. 4,391,909 to Lim et al describes a method in which cells are suspended in sodium alginate in saline, and droplets containing cells are produced. Droplets of cell-containing alginate flow into calcium chloride in saline. The negatively charged alginate droplets bind calcium and form a calcium alginate gel. The microcapsules are washed in saline and incubated with poly-L-lysine or poly-L-ornithine (or combinations thereof); the positively charged poly-l-lysine and/or poly-L-ornithine displaces calcium ions and binds (ionic) negatively charged alginate, producing an outer poly-electrolyte semipermeable membrane. An exterior coating of sodium alginate may be added by washing the microcapsules with a solution of sodium alginate, which ionically bonds to the poly-L-lysine and/or poly-L-ornithine layer (this serves to reduce any inflammatory response that may be provoked in the subject by contact of the polycationic membrane to tissue). This technique produces what has been termed a “single-wall” microcapsule. A “double-wall” microcapsule can be produced by following the same procedure as for single-wall microcapsules, but prior to any incubation with sodium citrate, the microcapsules are again incubated with poly-l-lysine and sodium alginate.

In additional non-limiting examples of encapsulation methods, Chang et al., U.S. Pat. No. 5,084,350 discloses microcapsules enclosed in a larger matrix, where the microcapsules are liquefied once the microcapsules are within the larger matrix. Tsang et al., U.S. Pat. No. 4,663,286 discloses encapsulation using an alginate polymer, where the gel layer is cross-linked with a polycationic polymer such as polylysine, and a second layer formed using a second polycationic polymer (such as polyornithine); the second layer can then be coated by alginate. U.S. Pat. No. 5,762,959 to Soon-Shiong et al. discloses a microcapsule having a solid (non-chelated) alginate gel core of a defined ratio of calcium/barium alginates, with polymer material in the core. U.S. Pat. Nos. 5,801,033 and 5,573,934 to Hubbell et al. describe alginate/polylysine microspheres having a final polymeric coating (e.g., polyethylene glycol (PEG)); Sawhney et al., Biomaterials 13:863 (1991) describe alginate/polylysine microcapsules incorporating a graft copolymer of poly-1-lysine and polyethylene oxide on the microcapsule surface, to improve biocompatibility; U.S. Pat. No. 5,380,536 describes microcapsules with an outermost layer of water soluble non-ionic polymers such as polyethylene(oxide). U.S. Pat. No. 5,227,298 to Weber et al. describes a method for providing a second alginate gel coating to cells already coated with polylysine alginate; both alginate coatings are stabilized with polylysine. U.S. Pat. No. 5,578,314 to Weber et al. provides a method for microencapsulation using multiple coatings of purified alginate. U.S. Pat. No. 5,693,514 to Dorian et al. reports the use of a non-fibrogenic alginate, where the outer surface of the alginate coating is reacted with alkaline earth metal cations comprising calcium ions and/or magnesium ions, to form an alkaline earth metal alginate coating. The outer surface of the alginate coating is not reacted with polylysine. U.S. Pat. No. 5,846,530 to Soon-Shiong describes microcapsules containing cells that have been individually coated with polymerizable alginate, or polymerizable polycations such as polylysine, prior to encapsulation.

When desired, the alginate-polylysine microcapsules can be incubated in sodium citrate to solubilize any calcium alginate that has not reacted with poly-l-lysine, i.e., to solubilize the internal core of sodium alginate containing the cells, thus producing a microcapsule with a liquefied cell-containing core portion. See Lim and Sun, Science 210:908 (1980). Such microcapsules are referred to herein as having “chelated”, “hollow” or “liquid” cores.

When desired, the microcapsules may be treated or incubated with a physiologically acceptable salt such as sodium sulfate or like agents, in order to increase the durability of the microcapsule, while retaining or not unduly damaging the physiological responsiveness of the cells contained in the microcapsules. See, e.g., U.S. Pat. No. 6,783,964 to Opara.

One currently preferred method for the production of microcapsules is described in O. Khanna et al., Synthesis of multilayered alginate microcapsules for the sustained release of fibroblast growth factor-1 J. Biomed. Mater. Res. Part A: 95A: 632-640 (2010).

According to some embodiments of the present invention, a microcapsule comprises, consists of, or consists essentially of (i) a liquid aqueous or hydrogel core, (ii) a semipermeable membrane surrounding the core; and (iii) optionally live mammalian cells in the core. In certain embodiments of the present invention, a microcapsule comprises one or more fibroblast growth factors and optionally one or more biologically active compounds. In particular embodiments of the present invention, a microcapsule further comprises an exterior sodium alginate coating over the semipermeable membrane, optionally comprising a fibroblast growth factor and/or one or more biologically active compounds. In certain embodiments of the present invention, a microcapsule comprises an anticoagulant, such as, but not limited to, heparin. In other embodiments of the present invention, a microcapsule is substantially free of an anticoagulant, such as, but not limited to, heparin.

“Substantially free” as used herein in reference to the presence of an anticoagulant in a microcapsule means that no anticoagulant is present in the microcapsule and/or a minimal amount of anticoagulant is present in the microcapsule such that the presence of the anticoagulant does not decrease the activity of a biologically active compound, such as, but not limited to, a fibroblast growth factor, by more than about 50% compared to the activity of the biologically active compound in a microcapsule with no anticoagulant present. In some embodiments of the present invention, no anticoagulant is added during the formation of a microcapsule. In other embodiments of the present invention, an anticoagulant can be partially or fully removed from a microcapsule before, after, and/or during the addition of a biologically active compound to the microcapsule.

In a particular embodiments of the present invention, a microcapsule is provided comprising, consisting essentially of, or consisting of: (i) a liquid aqueous or hydrogel core; (ii) a semipermeable membrane surrounding the core; (iii) an exterior sodium alginate coating; (iv) live mammalian pancreatic islet cells in the core; and (iv) a fibroblast growth factor, such as, but not limited to, FGF-1 and/or FGF-2, encapsulated in the exterior sodium alginate coating, wherein the microcapsule is optionally substantially free of an anticoagulant (e.g., heparin). Encapsulation of a fibroblast growth factor and/or a biologically active compound can be achieved by adding FGF and/or a biologically active compound to the sodium alginate solution prior to forming the exterior coating.

Microcapsules may be of any suitable size, such as from 10, 20 or 30 microns in diameter, up to 1000, 2000, or 5000 microns in diameter. Microcapsules may contain any suitable amount of cell. For example, in some embodiments, the cells are included in the microcapsules in an amount of from 1,000 or 2,000 cells per microcapsule up to 1×10⁶, 1×10⁸, or 1×10⁹ cells per microcapsule; and the cells are included in the microcapsules an amount of from 1,000 or 2,000 cells per microcapsule up to 1×10⁶, 1×10⁸, or 1×10⁹ cells per microcapsule.

The microcapsules of the present invention can further optionally comprise an oxygen-generating particle in the core of the microcapsule. In particular embodiments of the present invention, oxygen-generating particle can be present in a microcapsule of the present invention in an amount sufficient to lengthen the duration of viability of the mammalian cells in the microcapsule.

As described in U.S. Patent Provisional Application Nos. 61/521,420 and 61/601,780, which are incorporated herein by reference in their entirety, any suitable oxygen-generating particle can be used including but not limited to encapsulated hydrogen peroxide, inorganic peroxides, or peroxide adducts such as described in US Patent Application Publication Nos. 2009/0169630 to Ward et al. and 2010/0112087 to Harrison et al. (the disclosures of which are incorporated by reference herein in their entirety). The oxygen-generating particles preferably comprise an organic or inorganic peroxide such as urea peroxide, calcium peroxide, magnesium peroxide, and/or sodium percarbonate. The oxygen-generating active agent is included in the composition in any suitable amount (e.g., from 0.1 or 1 to 10, 20, or 30 percent by weight, or more). In some embodiments calcium peroxide is preferred as it releases oxygen at a desireable rate in situ. The oxygen-generating active agent can be included in the polymer in solid form, such as in the form of a plurality of solid particles thereof.

In some embodiments a radical trap or peroxide or radical decomposition catalyst is also included in the oxygen-generating particle and/or the microcapsule composition (e.g., in an amount of from 0.1 or 1 to 10, 20 or 30 percent by weight, or more). Suitable examples of radical traps or decomposition catalysts include, but are not limited to, iron (including, but not limited to, iron particles or nanoparticles, enzymes such as catalase, peroxidase, or dehydrogenase (see, e.g., U.S. Pat. No. 7,189,329), compounds such as cyclic salen-metal compounds that have superoxide and/or catalase and/or peroxidase activity (see, e.g., U.S. Pat. No. 7,122,537), etc.). The radical trap or decomposing catalyst may be included in solid form (e.g., solid particulate form) and can be coated on or incorporated in the polymer, or both coated on and incorporated in the polymer).

In further embodiments of the present invention, an antioxidant is also included in the microcapsule (e.g., in an amount of from 0.1 or 1 to 10, 20 or 30 percent by weight, or more). Suitable examples of antioxidants include, but are not limited to, ascorbic acid or vitamin C, tocopherols and tocotrienols such as vitamin E and analogs thereof such as 6-hydroxy-2,5,7,8-tetramethylchroman-2-carboxylic acid (also known as “TROLOX”), porphyrin antioxidants, particularly manganese porphyrin superoxide dismutase/catalase mimetics such as Mn (III) tetrakis(N-ethylpyridinium-2-yl) porphyrin (MnTE-2-PyP) (see, e.g., R. Rosenthal et al., J. Biol. Inorg. Chem. 14: 979-991 (2009)), phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen, etc., including combinations thereof, See, e.g., U.S. Pat. Nos. 7,999,003 and 5,928,654.

Microcapsules of the present invention may be administered after production, refrigerated and/or cryopreserved for subsequent use, and/or cultured for subsequent use, as desired. Microcapsules of the invention may be washed (e.g., in sterile physiological saline solution) prior to formulation and/or administration, as needed depending upon their manner of production.

### 3. Additional Methods of Administration, Formulation and Uses

A further aspect of the present invention comprises methods of administering a microcapsule to a subject by contacting the microcapsule to an omentum pouch. In some embodiments of the present invention, the microcapsule is a microcapsule of the present invention. “Contacting” as used herein, refers to placing, dropping, submerging, injecting, and the like, microcapsules into and/or onto an omentum pouch.

“Omentum pouch” as used herein refers to a structure or housing comprising, consisting essentially of, or consisting of omentum that forms a partially or fully enclosed cavity. An omentum pouch can exist and/or be formed on and/or in an omentum, wherein the cavity is at least in part formed by omentum (e.g., a wall of the omentum pouch comprises omentum). The cavity can comprise components in addition to the microcapsules, such as, but not limited to, a fluid, gas, and/or tissue. An omentum pouch can comprise, consist essentially of, or consist of omentum from a subject of the methods of the present invention. Thus, an omentum pouch can be present in and/or can be formed from the omentum of a subject of the present invention.

Omentum is a peritoneal fold that comprises connective tissue and fat. In some embodiments of the present invention, an omentum pouch can be present in a subject as a result of the native structure and/or folding of the omentum. The omentum pouch can be made from the greater omentum, which extends from the stomach, and/or from the lesser omentum, which extends from the liver. In particular embodiments of the present invention, an omentum pouch comprises the greater omentum. Thus, in contrast to implanting microcapsules into the peritoneal cavity, which is the space between the parietal peritoneum and visceral peritoneum, the microcapsules are contacted directly onto and/or into omentum.

An omentum pouch can be formed by any suitable method. Generally, forming an omentum pouch requires access to and/or exposure of a subject's omentum using a surgical method, such as but not limited to, a laparotomy. An omentum pouch can be formed before, after and/or during the step of contacting a microcapsule to an omentum. In some embodiments of the present invention, after contacting a microcapsule to an omentum, portions of the omentum and/or another tissue can be used to cover, hold, and/or enclose the microcapsule and thus form an omentum pouch with the microcapsules located in the cavity formed by the omentum and/or other tissue. In other embodiments of the present invention, an omentum pouch is first formed by using omentum and/or another tissue to form a partially or fully enclosed cavity, and then a microcapsule can be contacted to the omentum pouch, such as, but not limited to, by placing and/or injecting the microcapsule into the cavity of the omentum pouch. In particular embodiments of the present invention, an omentum pouch consists of omentum and the cavity is fully enclosed by omentum. Upon forming an omentum pouch, the omentum and/or other tissue can be held together using a surgical glue, such as, but not limited to, fibrin glue, or by suturing portions of the omentum and/or other tissue together. Alternatively, an omentum pouch can be formed by directly injecting microcapsules into an omentum. Further exemplary methods of forming an omentum pouch include, but are not limited to, those described in U.S. Patent Application Publication Nos. 2011/0274666, 2010/0316690, Berman et al. American Journal of Transplantation, 9: 91-104 (2009), McQuilling et al. Transplant. Proc. 43(9):3262-4 (2011), Opara et al. J. of Investig. Med. 58(7):831-7 (2010), Moya et al. J. Surg. Res. 160(2):208-12 (2010), and Moya et al. Microvasc. Res. 78(2):142-7 (2009), the contents of which are incorporated herein by reference in their entirety.

According to one aspect of the present invention, a method of administering live mammalian cells to a subject is provided, the method comprising contacting a microcapsule to an omentum pouch in a subject, wherein the microcapsule comprises live mammalian cells, thereby administering live mammalian cells to the subject. In particular embodiments of the present invention, the microcapsule comprises: (i) a liquid aqueous or hydrogel core comprising the live mammalian cells; (ii) a semipermeable membrane surrounding the core; and optionally (iii) an exterior sodium alignate coating.

A further aspect of the present invention comprises a method of implanting a microcapsule in a subject, the method comprising: (a) contacting a microcapsule to a portion of a subject's omentum, (b) forming an omentum pouch in the subject that at least partially surrounds the microcapsule, and (c) implanting the omentum pouch comprising the microcapsule into the subject. “Implanting” as used herein refers to inserting, transplanting, grafting, and the like, the omentum pouch into the subject. An omentum pouch can be implanted′ into the same location or a similar location in the subject compared to the location of the omentum used to form the omentum pouch prior to formation. Alternatively, an omentum pouch can be implanted into a different location in a subject, such as, but not limited to, into, onto, and/or next to a different tissue (e.g., a muscle), compared to the location of the omentum used to form the omentum pouch prior to formation.

According to the methods of the present invention, microcapsules are formulated for contact with an omentum pouch. Formulation of the microcapsules can comprise mixing the microcapsules with a pharmaceutically acceptable carrier and/or excipient, such as by mixing the microcapsules with sterile physiological saline solution.

Further embodiments of the present invention comprise a method of treating diabetes in a subject in need thereof, the method comprising: (a) contacting a microcapsule to an omentum pouch in a subject, wherein the microcapsule comprises live mammalian cells, and (b) implanting the omentum pouch comprising the microcapsule into the subject. In particular embodiments of the present invention, the live mammalian cells are pancreatic islet cells.

In certain embodiments of the present invention, a method of enhancing body weight gain in a subject is provided, the method comprising: (a) contacting a microcapsule to an omentum pouch in a subject, and (b) implanting the omentum pouch comprising the microcapsule into the subject. In some embodiments, of the present invention the microcapsule comprises: (i) a liquid aqueous or hydrogel core, (ii) a semipermeable membrane surrounding the core, (iii) an exterior sodium alginate coating, (iv) optionally live mammalian pancreatic islet cells in the core, and (iv) a fibroblast growth factor (e.g., FGF-1 and/or FGF-2) encapsulated in the exterior sodium alginate coating, wherein the microcapsule is optionally substantially free of heparin.

In other embodiments of the present invention, a method of enhancing myogenesis in a subject is provided, the method comprising: (a) contacting a microcapsule to an omentum pouch in a subject, and (b) implanting the omentum pouch comprising the microcapsule into the subject. In some embodiments, of the present invention the microcapsule comprises: (i) a liquid aqueous or hydrogel core, (ii) a semipermeable membrane surrounding the core, (iii) an exterior sodium alginate coating, (iv) optionally live mammalian pancreatic islet cells in the core, and (iv) a fibroblast growth factor (e.g., FGF-1 and/or FGF-2) encapsulated in the exterior sodium alginate coating, wherein the microcapsule is optionally substantially free of heparin.

The present invention is explained in greater detail in the following non-limiting Examples.

### Example 1

Immunoisolation by microencapsulation is a strategy designed to overcome the two major barriers to routine islet transplantation, namely: limited supply of human organs and the need to use immunosuppressive drugs to prevent graft rejection. However, the ideal site for engraftment of encapsulated islets has not been established. Microencapsulated islets have been transplanted into the general peritoneal cavity, but with variable success and an inability to recover islet grafts for analysis. The purpose of our study was to determine the viability of encapsulated islet allografts in an alternative site, the omentum pouch, made in immune-competent diabetic rats.

Methods:

Islets isolated from Wistar-Furth rats were encapsulated in microcapsules (300-400 μm in diameter) made with 1.5 wt % ultrapurified high M alginate (LVM) and crosslinked with 100 mM CaCl₂ solution. Following perm-selective coating with 0.1 wt % Poly-L-Ornithine, the inner LVM core of the microcapsules was chelated (liquefied) with 55 mM of sodium citrate for 2 min prior to a final coating with high G alginate (1.25 wt % ultrapurified LVG). The microcapsules were then rinsed with a mixture of 22 mM CaCl₂ and 0.9% NaCl prior to use in experiments. Owing to limited omental tissue space in the rat, a marginal mass of the encapsulated islets (˜2000 islets/kg) was transplanted in an omentum pouch made in each of 5 STZ-diabetic Lewis rats whose blood glucose, plasma C-peptide, and body weights were monitored for 90 days along with those of a control group (n=5) which received empty capsules (no islets). The control group received daily insulin injections to keep blood glucose <500 mg/dL during follow-up.

Results:

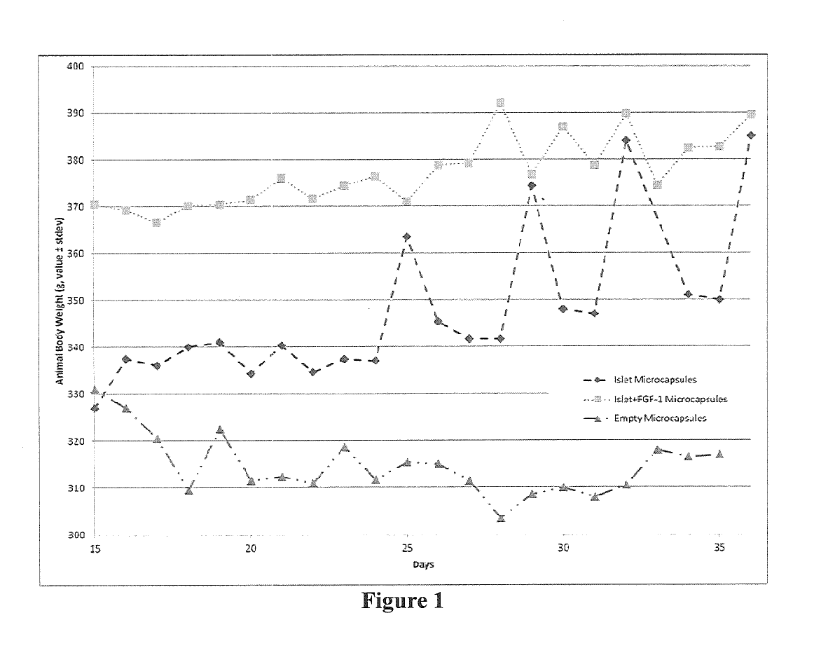

Although normoglycemia was not achieved with the marginal mass, the islet recipients had a 12% reduction in their mean blood sugar levels compared to controls (p<0.001), and increased their body weight from the diabetic baseline in contrast to the control group (see, e.g., FIG. 1). Also, C-Peptide (Mercodia ELISA kit) increased from a non-detectable level to a range of 200-600 pmol/L in the islet recipients, but not in the control group during the 3-month period.

These data show for the first time that a marginal mass of encapsulated islet transplants have long-term function in an omentum pouch making it a possible alternative site for encapsulated islet transplantation in large animals and humans with abundant omental tissue.

### Example 2

The onset of Type 1 diabetes is accompanied by a progressive decrease in body weight, which is mitigated to some extent by insulin administration. However, although fibroblast growth factor-1 (FGF-1) is a known mitogen, its effect on body weight has not been previously described.

Methods:

We prepared 3 groups of alginate microcapsules (300-400 μm in diameter) made with 1.5 wt % ultrapurified high M alginate (LVM). Two groups of these microcapsules contained islets isolated from Wistar Furth rats. Following perm-selective coating with 0.1 wt % Poly-L-Ornithine, the microcapsules were finally coated with high G alginate (1.25 wt % LVG), which was supplemented with 1.794 μg FGF-1/100 microcapsules in one of the two islet-containing groups. The inner LVM core of all three groups of microcapsules was chelated with 55 mM of sodium citrate for 2 min. Because of tissue size limitation in the rat, a marginal mass of encapsulated islets (˜2000 islets/kg) from group 1 (no FGF-1) and group 2 (FGF-1 supplemented) was transplanted in an omentum pouch made in each of 5 STZ-diabetic Lewis rats whose blood glucose, plasma C-peptide and body weights were monitored for one month along with those of the control group receiving empty microcapsule transplants (no islets and no FGF-1, n=5). Group 3 animals received daily insulin injections to keep blood glucose <500 mg/dL during the follow-up period.

Results:

Blood glucose levels were significantly different among the 3 groups with group 1 having the lowest level (see, e.g., FIG. 1). The mean+SD plasma C-Peptide levels measured at 1 month in group 3 was at the limit of detection by Mercodia ELISA, while it was higher in group 1 than group 2 (379+29 vs 114+28 pmol/L, p<0.05). After 1 month, group 3 had the lowest mean body weight (329.2+26.6 g) among the 3 groups, and despite the higher insulin level as represented by C-peptide levels in group 1, the FGF-1 supplemented islet transplant recipients (group 2) gained more weight than group 1 (61.7+12.6 vs 33.2+18.6 g, p<0:05).

These data suggest that FGF-1 treatment may enhance body weight gain in diabetes.

### Example 3

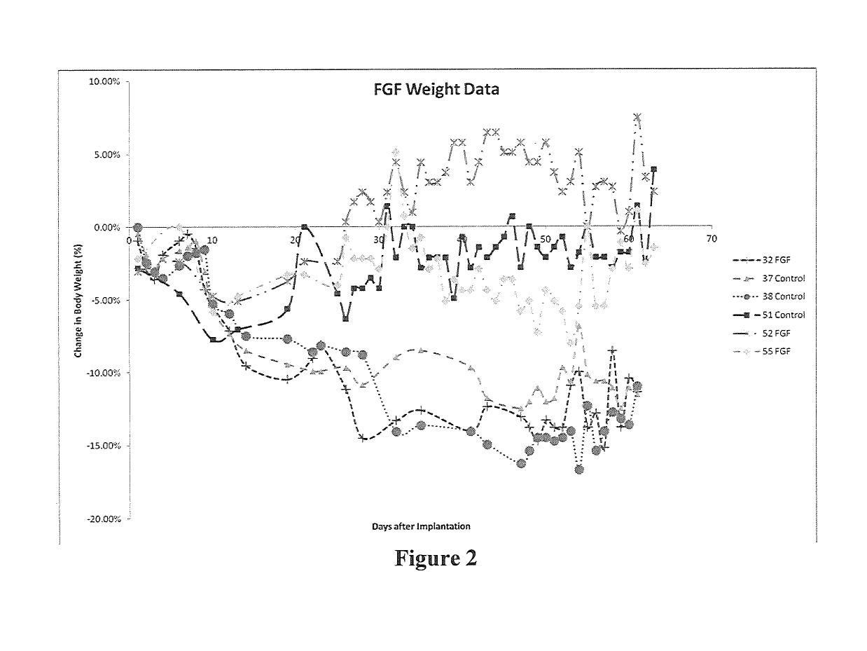

FGF-1 was obtained from Peprotech, Princeton Business Park, 5 Crescent Avenue, P.O. Box 275m Rocky Hill, N.J. 08553, United States (Cat #100-17A, Lot #1206C707 12809). From the stock solution a working solution of 270 μg/mL was made with 5 mM sodium phosphate and 0.1% BSA. The FGF-1 working solution was mixed with 1.25% LVG to form a solution containing 3 μg/FGF-1 with 5 U/mL Heparin prior to incubating with PLO-coated alginate microbeads in order to entrap the FGF-1 and heparin in the outer alginate layer. For the protein entrapment in the outer layer, the LVG-FGF-1-Heparin solution was mixed with the PLO-coated alginate microcapsules for 45 minutes prior to washing three times with 0.9% Saline and transplantation in the omentum pouches created in STZ-diabetic rats. Microcapsules that contained no islets and no protein as well as microcapsules that contained no islets but only protein were transplanted into omentum pouches of the diabetic rats as controls. Following transplantation, these control diabetic rats were treated by daily insulin injections, and body weight measurements were routinely measured in all animals. Results are given in FIG. 2. The data show that 2 out of 3 diabetic rats transplanted with FGF-1 with no islets had better weight gain than 2 of 3 control rats that were transplanted with empty microcapsules containing NO islets and NO protein, and thereby suggest that FGF-1 treatment may be useful in enhancing weight gain in other non-diabetic conditions requiring no insulin treatment.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

1. A method of enhancing weight gain in a subject in need thereof, the method comprising:
administering said subject a fibroblast growth factor (FGF) in an amount effective to enhance weight gain in said subject.

2. The method of claim 1, wherein the fibroblast growth factor comprises fibroblast growth factor-1 (FGF-1) and/or growth factor-2 (FGF-2).

3. The method of claim 1, wherein said subject is not afflicted with diabetes.

4. The method of claim 1, wherein said subject is afflicted with diabetes.

5. The method of claim 1, further comprising concurrently administering said subject insulin in a treatment-effective amount.

6. The method of claim 1, wherein said administering step is carried out by administering microcapsules in said subject, said microcapsules comprising said FGF, and optionally live mammalian islet cells.

7. The method of claim 6, wherein the microcapsule further comprises a liquid aqueous or hydrogel core comprising the live mammalian islet cells and a semipermeable membrane surrounding the core.

8. The method of claim 6, wherein the microcapsule further comprises an exterior coating, said coating comprising a biodegradable polymer.

9. The method of claim 8, wherein said exterior coating further comprises at least one biologically active compound.

10. The method of claim 9, wherein said at least one biologically active compound comprises said FGF.

11. The method of claim 9, wherein said at least one biologically active compound comprises an anticoagulant.

12. The method of claim 1, wherein said administering step is carried out by intraperitoneal, intramuscular, or subcutaneous injection.

13. The method of claim 6, wherein said administering step is carried out by implanting or injecting said microcapsules into an omentum pouch in said subject.

14. (canceled)
